(19) United States
(12) Patent Application Publication
(10) Pub. No.: US 2023/0329673 A1
(43) Pub. Date: Oct. 19, 2023

(54) ULTRASOUND PROBE, ULTRASOUND ENDOSCOPE, LAMINATED BODY, AND ULTRASOUND PROBE MANUFACTURING METHOD

(71) Applicant: OLYMPUS MEDICAL SYSTEMS CORP., Tokyo (JP)

(72) Inventor: Satoshi YOSHIDA, Kawagoe-shi (JP)

(73) Assignee: OLYMPUS MEDICAL SYSTEMS CORP., Tokyo (JP)

(21) Appl. No.: 18/211,811

(22) Filed: Jun. 20, 2023

Related U.S. Application Data
(63) Continuation of application No. PCT/JP2021/009508, filed on Mar. 10, 2021.

(51) Int. Cl. A61B 8/00; A61B 8/12; B06B 1/06; H10N 30/088

(52) U.S. Cl. CPC A61B 8/4483; A61B 8/12; B06B 1/067; B06B 1/0681; H10N 30/088; B06B 2201/76

(57) ABSTRACT

An ultrasound probe includes: an ultrasound transducer that includes a piezoelectric element configured to transmit and receive ultrasound waves to and from a subject; and an adjustment layer that is laminated on the piezoelectric element, the adjustment layer being provided with a cut surface that is cut by a blade configured to cut the piezoelectric element, the adjustment layer including an adjustment material configured to improve cutting performance of the blade.

### CROSS-REFERENCE TO RELATED APPLICATION

This application is a continuation of International Application No. PCT/JP2021/009508, filed on Mar. 10, 2021, the entire contents of which are incorporated herein by reference.

### BACKGROUND

### 1. Technical Field

The present disclosure relates to an ultrasound probe, an ultrasound endoscope, a laminated body, and an ultrasound probe manufacturing method.

### 2. Related Art

In the related art, an ultrasound probe that includes a plurality of ultrasound transducers that are formed by cutting a piezoelectric element by a blade is known (for example, Japanese Laid-open Patent Publication No. 2001-46368).

Further, in a cutting process using the blade, in some cases, abrasive grains of the blade may be covered by foreign matters and clogging may occur, or abrasive grains of the blade may be scraped such that a surface of the blade is flattened and glazing may occur, so that the blade may be deteriorated. If the blade is deteriorated, an excessive load is applied to a work that is a processing target and the work may be damaged.

Therefore, to prevent damage of the work, a technique of performing a certain process, such as dressing or pre-cutting, on the blade to improve cutting performance of the deteriorated blade has been used. In the dressing, an outer circumference of the blade is scraped to expose a new cutting surface in order to improve the cutting performance of the blade. In the pre-cutting, abrasive grains embedded in adhesives that hold the abrasive grains are exposed for dressing in order to improve the cutting performance of the blade. If the cutting performance of the deteriorated blade is improved by the technique as described above, an yield rate of an ultrasound probe that is manufacture by processing the work is improved.

### SUMMARY

In some embodiments, an ultrasound probe includes: an ultrasound transducer that includes a piezoelectric element configured to transmit and receive ultrasound waves to and from a subject; and an adjustment layer that is laminated on the piezoelectric element, the adjustment layer being provided with a cut surface that is cut by a blade configured to cut the piezoelectric element, the adjustment layer including an adjustment material configured to improve cutting performance of the blade.

In some embodiments, an ultrasound endoscope includes: the ultrasound probe; an insertion portion including a distal end on which the ultrasound probe is arranged, the insertion portion being configured to be inserted into the subject; and an operating portion that is arranged on a proximal end side of the insertion portion.

In some embodiments, a laminated body includes: a piezoelectric layer that is made of a piezoelectric material; and an adjustment material layer that is laminated on the piezoelectric layer and that includes an adjustment material configured to improve cutting performance of a blade configured to cut the piezoelectric layer.

In some embodiments, an ultrasound probe manufacturing method includes: preparing a laminated body including a piezoelectric layer that is made of a piezoelectric material, and an adjustment material layer that is laminated on the piezoelectric layer and that includes an adjustment material configured to improve cutting performance of a blade configured to cut the piezoelectric layer; and cutting the piezoelectric layer and at least a part of the adjustment material layer by the blade.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an endoscope system including an ultrasound probe according to a first embodiment;

FIG. 2 is a perspective view of the ultrasound probe according to the first embodiment;

FIG. 3 is a diagram viewed from an arrow A in FIG. 2;

FIG. 4 is a diagram viewed from an arrow B in FIG. 2;

FIG. 5 is a diagram illustrating a state in which a laminated body is cut by a blade;

FIG. 6 is a diagram illustrating a state in which the laminated body is cut by the blade;

FIG. 7 is a side view of an ultrasound probe according to a second embodiment;

FIG. 8 is a side view of the ultrasound probe according to the second embodiment;

FIG. 9 is a side view of an ultrasound probe according to a third embodiment;

FIG. 10 is a side view of the ultrasound probe according to the third embodiment;

FIG. 11 is a side view of an ultrasound probe according to a fourth embodiment;

FIG. 12 is a side view of the ultrasound probe according to the fourth embodiment;

FIG. 13 is a diagram illustrating a state in which an ultrasound probe according to a first modification is manufactured;

FIG. 14 is a diagram illustrating a state in which the ultrasound probe according to the first modification is manufactured;

FIG. 15 is a diagram illustrating a state in which the ultrasound probe according to the first modification is manufactured;

FIG. 16 is a diagram illustrating a state in which an ultrasound probe according to a second modification is manufactured;

FIG. 17 is a diagram illustrating a state in which an ultrasound probe according to a third modification is manufactured;

FIG. 18 is a diagram illustrating a state in which the ultrasound probe according to the third modification is manufactured;

FIG. 19 is a diagram illustrating a state in which an ultrasound probe according to a fourth modification is manufactured; and

FIG. 20 is a diagram illustrating a state in which the ultrasound probe according to the fourth modification is manufactured.

### DETAILED DESCRIPTION

Embodiments of an ultrasound probe, an ultrasound endoscope, a laminated body, and an ultrasound probe manufacturing method according to the disclosure will be described below with reference to the drawings. The disclosure is not limited by the embodiments below. In the embodiments described below, an ultrasound probe that includes an acoustic matching layer, a dematching layer, a backing layer, and the like, an ultrasound endoscope, and an ultrasound probe manufacturing method will be explained as examples; however, the disclosure is applicable to a general ultrasound probe including a piezoelectric element, a general ultrasound endoscope, and a general ultrasound probe manufacturing method. Similarly, the disclosure is applicable to a general laminated body including a piezoelectric layer.

Further, in description of the drawings, the same or corresponding components are appropriately denoted by the same reference symbols. Furthermore, it is necessary to note that the drawings are schematic, and dimensional relations among the components, ratios among the components, and the like may be different from actual ones. Moreover, the drawings may include portions that have different dimensional relations or ratios.

### First Embodiment

Overall Configuration of Endoscope System

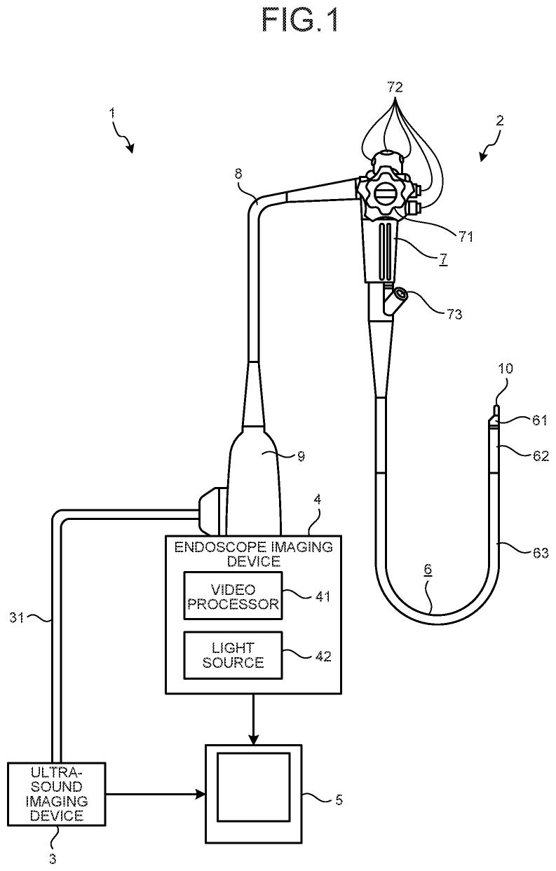

FIG. 1 is a schematic diagram illustrating an endoscope system that includes an ultrasound probe according to a first embodiment. An endoscope system 1 is a system that performs ultrasound diagnosis and treatment inside a subject, such as a human being, by using an ultrasound endoscope. As illustrated in FIG. 1, the endoscope system 1 includes an ultrasound endoscope 2, an ultrasound imaging device 3, an endoscope imaging device 4, and a display 5.

The ultrasound endoscope 2 is configured such that a part of the ultrasound endoscope 2 can be inserted into a subject and has a function to transmit ultrasound pulses (acoustic pulses) to a body wall inside the subject, receive ultrasound echoes reflected by the subject, and output echo signals and a function to capture an image inside the subject and output an image signal. Meanwhile, a detailed configuration of the ultrasound endoscope 2 will be described later.

The ultrasound imaging device 3 is electrically connected to the ultrasound endoscope 2 via an ultrasound cable 31, outputs a pulse signal to the ultrasound endoscope 2 via the ultrasound cable 31, and inputs an echo signal from the ultrasound endoscope 2. Further, in the ultrasound imaging device 3, a predetermined process is performed on the echo signal and an ultrasound image is generated.

An endoscope connector 9 (to be described later) of the ultrasound endoscope 2 is detachably connected to the endoscope imaging device 4. The endoscope imaging device 4 includes a video processor 41 and a light source 42.

The video processor 41 receives input of an image signal from the ultrasound endoscope 2 via the endoscope connector 9. Further, the video processor 41 performs a predetermined process on the image signal and generates an endoscopic image.

The light source 42 supplies illumination light for illuminating the inside of the subject to the ultrasound endoscope 2 via the endoscope connector 9.

The display 5 is configured using liquid crystal, organic electro luminescence (EL), cathode ray tube (CRT), or a projector, and displays the ultrasound image that is generated by the ultrasound imaging device 3, the endoscopic image that is generated by the endoscope imaging device 4, or the like.

Configuration of Ultrasound Endoscope

A configuration of the ultrasound endoscope 2 will be described below. The ultrasound endoscope 2 includes an insertion portion 6, an operating portion 7, a universal cord 8, and an endoscope connector 9.

The insertion portion 6 is a portion that is inserted into the subject. The insertion portion 6 includes an ultrasound probe 10 that is arranged on a distal end side, a rigid member 61 that is connected to a proximal end side of the ultrasound probe 10, a bending portion 62 that is connected to a proximal end side of the rigid member 61 and that allows bend, a flexible tube 63 that is connected to a proximal end side of the bending portion 62 and that has flexibility.

The operating portion 7 is a portion that is connected to a proximal end side of the insertion portion 6 and receives various kinds of operation from a doctor or the like. The operating portion 7 includes a bending knob 71 that allows bending operation of the bending portion 62 and a plurality of operating members 72 for performing various kinds of operation. Further, a treatment tool insertion opening 73 for inserting a treatment tool is arranged in the operating portion 7.

The universal cord 8 is a cord that is extended from the operating portion 7, and in which a tube that constitutes a part of a light guide, a transducer cable, a signal cable, and a pipeline is arranged.

The endoscope connector 9 is arranged on an end portion of the universal cord 8. Further, the endoscope connector 9 is connected to the video processor 41 and the light source 42 when the ultrasound cable 31 is connected and inserted in the endoscope imaging device 4.

Configuration of Ultrasound Probe

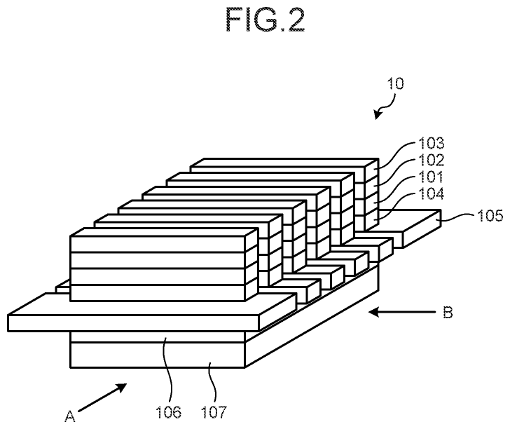

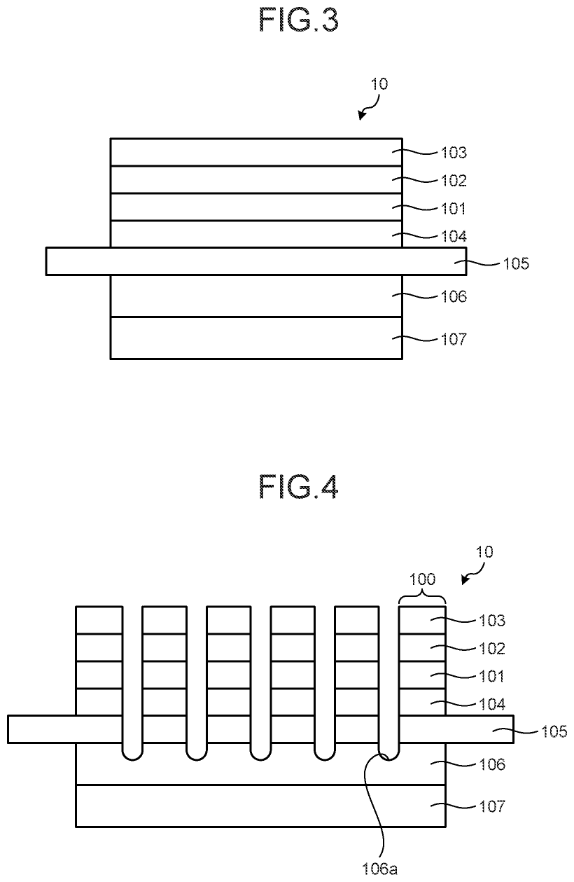

A configuration of the ultrasound probe 10 will be described below. FIG. 2 is a perspective view of the ultrasound probe according to the first embodiment. FIG. 3 is a diagram viewed from an arrow A in FIG. 2. FIG. 4 is a diagram viewed from an arrow B in FIG. 2.

As illustrated in FIG. 4, the ultrasound probe 10 includes a plurality of strip-shaped ultrasound transducers 100. Each of the ultrasound transducers 100 includes a piezoelectric element 101, a first acoustic matching layer 102, a second acoustic matching layer 103, a dematching layer 104, an FPC layer 105 as a substrate layer, an adjustment layer 106, and a backing layer 107. Meanwhile, it is sufficient that the ultrasound probe 10 is an ultrasound transducer including the piezoelectric elements 101 and any configuration of a convex type, a linear type, and a radial type is applicable.

Each of the piezoelectric elements 101 transmits and receives ultrasound waves to the subject. The piezoelectric element 101 may include a piezoelectric body that is made of a single crystal. Specifically, the piezoelectric element 101 is formed by using a piezoelectric material, such as a PMN-PT single crystal, a PMN-PZT single crystal, a PZN-PT single crystal, a PIN-PZN-PT single crystal, or a relaxer material. Meanwhile, the PMN-PT single crystal is an abbreviation of a lead magnesium niobate and lead titanate solid solution. The PMN-PZT single crystal is an abbreviation of a lead magnesium niobate and lead zirconate titanate solid solution. The PZN-PT single crystal is an abbreviation of a lead zinc niobate and lead titanate solid solution. The PIN-PZN-PT single crystal is an abbreviation of a lead indium niobate, lead zinc niobate, and lead titanate solid solution. The relaxer material is a generic term of a ternary system piezoelectric material that is obtained by adding lead zirconate titanate (PZT) to lead-based complex perovskite that is a relaxer material in order to increase a piezoelectric constant or permittivity. The lead-based complex perovskite is represented by Pb(B1, B2)O3, where B1 is any of magnesium, zinc, indium, and scandium, and B2 is any of niobium, tantalum, and tungsten. The piezoelectric materials as described above have good piezoelectric effects. Therefore, it is possible to reduce an electrical impedance value even if a device size is reduced, which is preferable from the viewpoint of impedance matching with an electrode.

The first acoustic matching layer 102 and the second acoustic matching layer 103 realize acoustic impedance matching between the piezoelectric element 101 and an observation target in order to effectively transmit sound (ultrasound waves) between the piezoelectric element 101 and the observation target. The first acoustic matching layer 102 and the second acoustic matching layer 103 are made of different materials. Meanwhile, in the first embodiment, explanation will be given based on the assumption that the two acoustic matching layers (the first acoustic matching layer 102 and the second acoustic matching layer 103) are provided, but it may be possible to provide a single layer or three or more layers depending on characteristics of the piezoelectric element 101 and the observation target. Further, the ultrasound probe 10 need not always include an acoustic matching layer.

The dematching layer 104 is laminated on the piezoelectric element 101 in a certain direction (downward direction in FIG. 3 and FIG. 4) opposite to a direction in which the piezoelectric element 101 transmits and receives ultrasound waves, is made of a material with higher acoustic impedance than the piezoelectric element 101, and reflects ultrasound waves that are generated by the piezoelectric element 101. Meanwhile, when the dematching layer 104 is arranged between the piezoelectric element 101 and the FPC layer 105, it is preferable to use a conductive material; however, if the dematching layer 104 is made of a material with low conductivity, it may be possible to electrically connect the piezoelectric element 101 and the FPC layer 105 by performing surface plating.

The FPC layer 105 is a flexibly substrate that include a wire that is electrically connected to the piezoelectric element 101.

The adjustment layer 106 is laminated on the piezoelectric element 101 in a certain direction (downward direction in FIG. 3 and FIG. 4) opposite to a direction in which the piezoelectric element 101 transmits and receives ultrasound waves. The adjustment layer 106 includes a cut surface 106a that is cut by a blade that cuts the piezoelectric element 101. The cut surface 106a is a surface that is formed by being cut by the blade and is, for example, a groove; however, the cut surface 106a may be a flat surface and the shape is not specifically limited.

Further, the adjustment layer 106 includes an adjustment material that improves cutting performance of the blade that cuts the piezoelectric element 101. Specifically, the adjustment material is a material for performing at least one of dressing and pre-cutting on the blade. Further, the adjustment layer 106 is laminated so as to come into contact with the FPC layer 105 and the backing layer 107.

If the adjustment material is a material for performing dressing on the blade, it is preferable that the adjustment material is an abrasive grain that is finer than an abrasive grain used in the blade. If the adjustment material is the abrasive grain that is finer than the abrasive grain used in the blade, it is possible to expose a new cut surface by cutting an outer circumference of the blade. Specifically, the adjustment layer 106 includes, as the adjustment material, an abrasive grain that is one grade smaller than the abrasive grain of the blade. The abrasive grain is, for example, a diamond abrasive grain, but is not limited to the diamond abrasive grain and may be any abrasive grain.

If the adjustment material is a material for performing pre-cutting on the blade, it is preferable that the adjustment material is an abrasive grain that is finer than the abrasive grain used in the blade and is made of a certain material that is harder than an adhesive used in the blade. If the adjustment material is the abrasive grain that is finer than the abrasive grain used in the blade and is made of a certain material that is harder than the adhesive used in the blade, it is possible to expose an abrasive grain that is embedded in the adhesive for holding the abrasive grain and realize dressing. Specifically, if the adhesive is nickel, it is sufficient that the adjustment material is a material that is harder than nickel and that is softer than the abrasive grain. It is preferable to appropriately select the adjustment material depending on a processing target, and, for example, if the processing target is the piezoelectric element 101, the adjustment material may be made of the same material as the piezoelectric element 101. Further, the adjustment material may be selected from a piezoelectric material, a silicon wafer, piezoelectric ceramics, machinable ceramics, tungsten carbide, or and the like depending on the processing target.

Furthermore, the adjustment material may be a material that is softer than the adhesive used in the blade. Specifically, if the adhesive is nickel, the adjustment material may be a material that is softer than nickel. It is sufficient that the adjustment material is a material, such as lead titanate powder, alumina powder, or a grass grain, that is harder than resin used in the backing layer 107 and the adjustment layer 106 and that is harder than an adhesive used to bond components included in the ultrasound transducer 100. In this case, it is possible to remove, by the adjustment material, resin that is attached to the blade or a highly malleable metal.

The backing layer 107 is made of a backing material that absorbs or attenuates unneeded ultrasound waves to prevent unneeded ultrasound waves that are generated by operation of the piezoelectric element 101 from being returned to elements. Specifically, the backing layer 107 is made of a material with a high attenuation rate, such as epoxy resin in which a filler, such as alumina or zirconia, is dispersed, or rubber in which the above-described filler is dispersed.

Ultrasound Probe Manufacturing Method

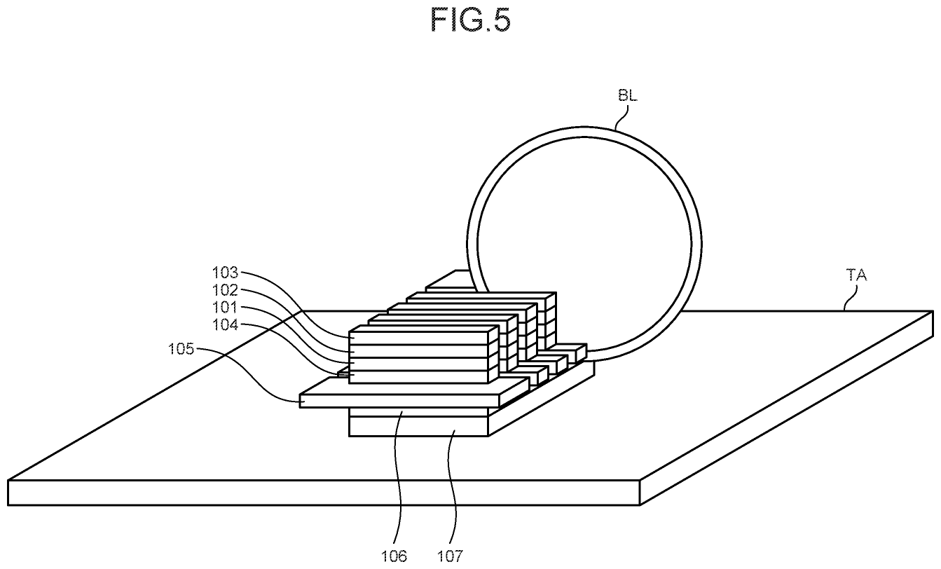

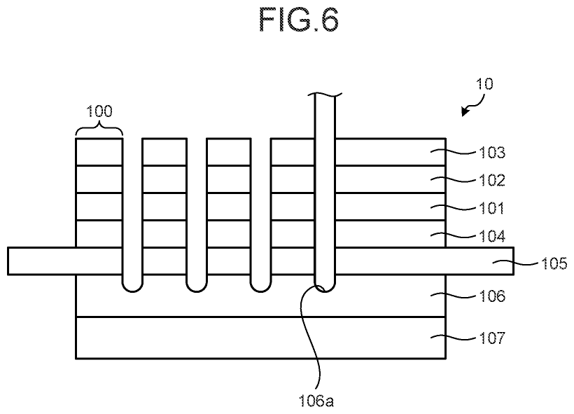

A method of manufacturing the ultrasound probe 10 will be described below. FIG. 5 and FIG. 6 are diagrams illustrating a state in which a laminated body is cut by the blade. FIG. 5 is a perspective view of the ultrasound probe 10 viewed from the same direction as FIG. 2, and FIG. 6 is a side view of the ultrasound probe 10 viewed from the same direction as FIG. 4.

First, a laminated body in which the piezoelectric elements 101 to the backing layer 107 are laminated is placed on a table TA. The laminated body includes a piezoelectric layer that is made of a piezoelectric material, and an adjustment material layer that is laminated on the piezoelectric layer and that includes the adjustment material for improving cutting performance of the blade that cuts the piezoelectric layer. Then, a blade BL is moved along a cutting direction (direction from left to right in FIG. 5) to perform cutting from the piezoelectric element 101 to a part of the adjustment layer 106 in the laminated body and form the plurality of ultrasound transducers 100. At this time, the blade BL cuts at least a part of the adjustment layer 106, so that dressing or pre-cutting is performed on the blade BL. Further, the cut surface 106a that is a groove is formed in the adjustment layer 106.

According to the first embodiment as described above, the adjustment material included in the adjustment layer 106 serves as a material for performing dressing or pre-cutting on the blade BL; therefore, when at least a part of the adjustment layer 106 is cut by the blade BL and the cut surface 106a is formed, dressing or pre-cutting is performed on the blade BL. As a result, the cutting performance of the blade BL is improved and a yield rate of the ultrasound probe 10 to be manufactured is improved. Further, it is not needed to replace a processing target work with a dressing board or a pre-cutting board; therefore, a movement distance of the blade BL in the cutting direction is not increased, so that it is possible to prevent an increase in a processing time and improve production efficiency.

In particular, if the piezoelectric element 101 is a piezoelectric body that is made of a single crystal, the piezoelectric element 101 is fragile and the ultrasound probe 10 is likely to be damaged during cutting; however, according to the ultrasound probe 10, the adjustment material included in the adjustment layer 106 performs dressing or pre-cutting on the blade BL, so that it is possible to prevent damage of the ultrasound probe 10 due to deterioration of the blade BL.

Furthermore, according to the first embodiment, the adjustment layer 106 is laminated so as to come into contact with the FPC layer 105 and the backing layer 107, and the adjustment material included in the adjustment layer 106 performs dressing or pre-cutting on the blade BL immediately after resin that is likely to cause clogging of the blade BL or metal of a highly malleable wire is cut, so that it is possible to increase the effect to improve the cutting performance of the blade BL.

### Second Embodiment

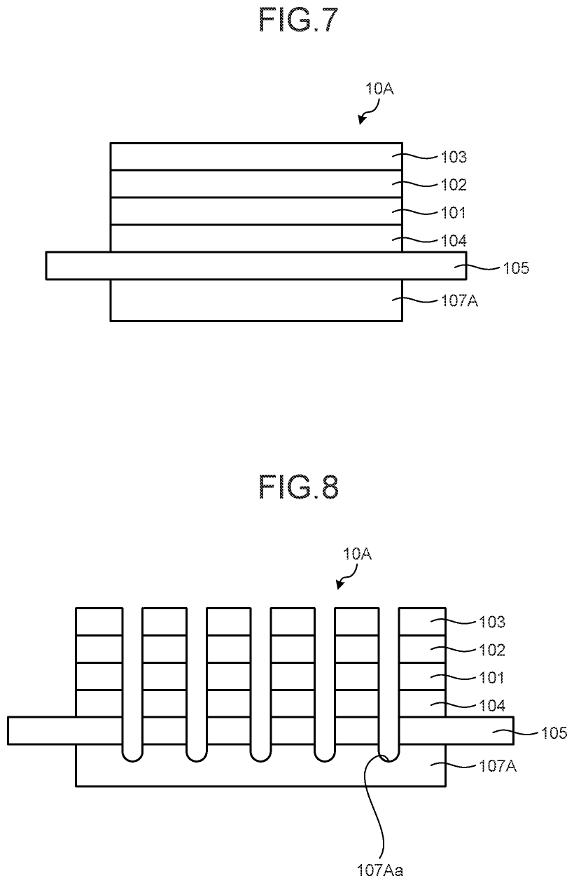

FIG. 7 and FIG. 8 are side views of an ultrasound probe according to a second embodiment. FIG. 7 is a diagram of an ultrasound probe 10A viewed from the same direction as FIG. 3, and FIG. 8 is a diagram of the ultrasound probe 10A viewed from the same direction as FIG. 4. A backing layer 107A is a backing layer that is made of a backing material that absorbs or attenuates ultrasound waves that are generated by the piezoelectric element 101, and includes an adjustment material that improves cutting performance of the blade that cuts the piezoelectric element 101. In other words, in the second embodiment, the adjustment layer is a backing layer that is made of a backing material that absorbs or attenuates ultrasound waves that are generated by the piezoelectric element 101. Further, the backing layer 107A includes a cut surface 107Aa that is cut by a blade that cuts the piezoelectric element 101.

According to the second embodiment, the adjustment material that is contained in the backing layer 107A serves as a material for performing dressing or pre-cutting on the blade; therefore, when at least a part of the backing layer 107A is cut by the blade and the cut surface 107Aa is formed, dressing or pre-cutting is performed on the blade. As a result, the cutting performance of the blade is improved and a yield rate of the ultrasound probe 10A to be manufactured is improved. Furthermore, it is not needed to replace a processing target work with a dressing board or a pre-cutting board; therefore, a movement distance of the blade in the cutting direction is not increased, so that it is possible to prevent an increase in a processing time and improve production efficiency.

Moreover, according to the second embodiment, the adjustment layer is the backing layer 107A that is made of resin, and the adjustment material included in the backing layer 107A performs dressing or pre-cutting on the blade at the same time as when resin that is likely to cause clogging of the blade is cut, so that it is possible to increase the effect to improve the cutting performance of the blade. Furthermore, the adjustment layer is laminated so as to come into contact with the FPC layer 105, and the adjustment material included in the backing layer 107A performs dressing or pre-cutting on the blade immediately after resin that is likely to cause clogging of the blade BL or metal of a highly malleable wire is cut, so that it is possible to increase the effect to improve the cutting performance of the blade.

Furthermore, according to the second embodiment, the backing layer 107A also functions as the adjustment layer, so that it is possible to reduce the number of layers to be laminated as compared to the first embodiment, reduce processes for lamination and bonding, and reduce variation in thicknesses at the time of manufacturing.

### Third Embodiment

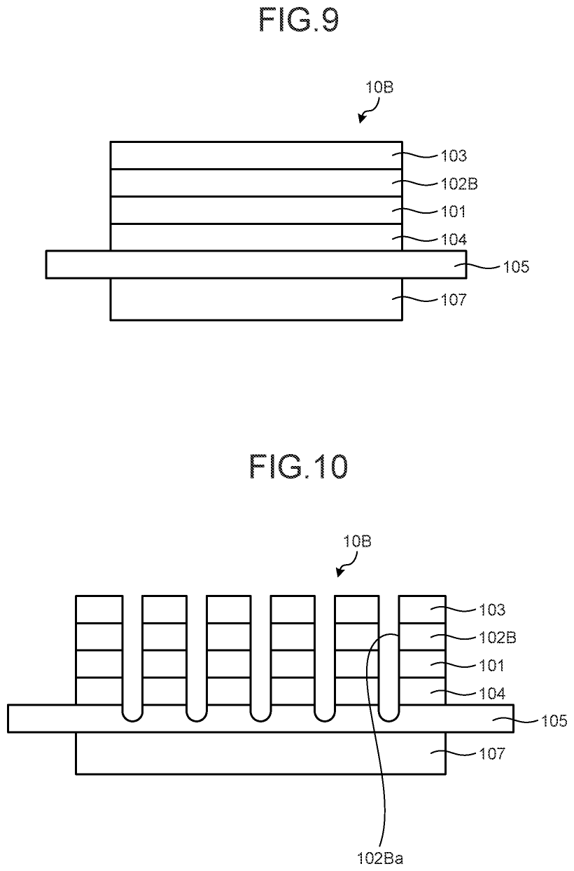

FIG. 9 and FIG. 10 are side views of an ultrasound probe according to a third embodiment. FIG. 9 is a diagram of an ultrasound probe 10B viewed in the same direction as FIG. 3, and FIG. 10 is a diagram of the ultrasound probe 10B viewed in the same direction as FIG. 4. A first acoustic matching layer 102B contains an adjustment material that realizes acoustic impedance matching between the piezoelectric element 101 and an observation target and improves cutting performance of a blade that cuts the piezoelectric element 101 in order to efficiently transmit sound (ultrasound waves) between the piezoelectric element 101 and the observation target. In other words, in the third embodiment, the adjustment layer is the first acoustic matching layer 102B that realizes acoustic impedance matching between the piezoelectric element 101 and the observation target in order to efficiently transmit sound (ultrasound waves) between the piezoelectric element 101 and the observation target. Further, the first acoustic matching layer 102B as the adjustment layer is laminated in a direction in which the piezoelectric element 101 transmits and receives ultrasound waves (upward direction in FIG. 9 and FIG. 10). Furthermore, the first acoustic matching layer 102B includes a cut surface 102Ba that is cut by a blade that cuts the piezoelectric element 101. The cut surface 102Ba is a surface that is formed by being cut by the blade and is a flat surface that extends along the side surface of the piezoelectric element 101.

According to the third embodiment, the adjustment material included in the first acoustic matching layer 102B serves as a material for performing dressing or pre-cutting on the blade; therefore, when the first acoustic matching layer 102B is cut by the blade and the cut surface 102Ba is formed, dressing or pre-cutting is performed on the blade. As a result, it is possible to improve the cutting performance of the blade and a yield rate of the ultrasound probe 10B to be manufactured. Furthermore, it is not needed to replace a processing target work with a dressing board or a pre-cutting board; therefore, a movement distance of the blade in the cutting direction is not increased, so that it is possible to prevent an increase in a processing time and improve production efficiency.

Moreover, according to the third embodiment, the adjustment layer is the first acoustic matching layer 102B that is made of resin; therefore, the adjustment material included in the first acoustic matching layer 102B performs dressing or pre-cutting on the blade at the same time as when resin that is likely to cause clogging of the blade is cut, so that it is possible to increase the effect to improve the cutting performance of the blade. Furthermore, the adjustment layer is laminated so as to come into contact with the second acoustic matching layer 103 that is made of resin, and the adjustment material included in the first acoustic matching layer 102B performs dressing or pre-cutting on the blade immediately after resin that is likely to cause clogging of the blade is cut, so that it is possible to increase the effect to improve the cutting performance of the blade.

Moreover, according to the second embodiment, the first acoustic matching layer 102B also functions as the adjustment layer, so that it is possible to reduce the number of layers to be laminated as compared to the first embodiment, reduce processes for lamination and bonding, and reduce variation in thicknesses at the time of manufacturing.

### Fourth Embodiment

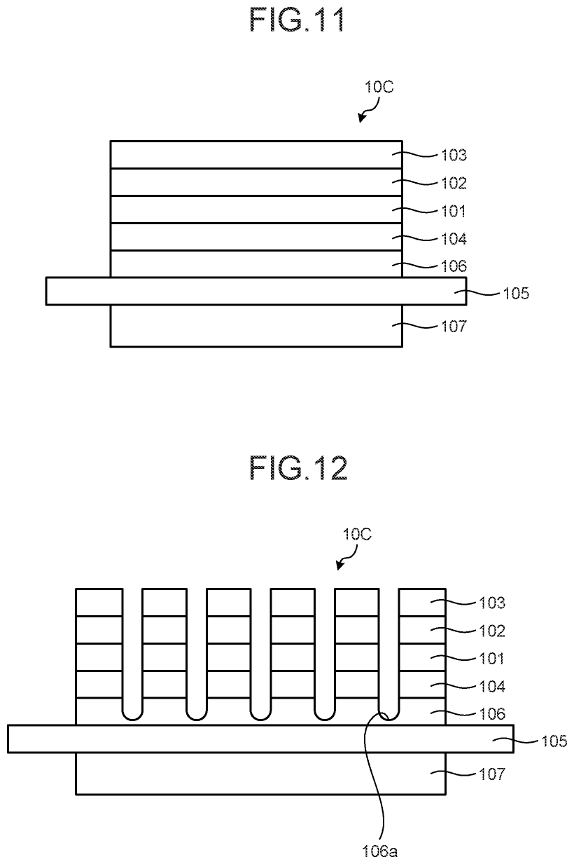

FIG. 11 and FIG. 12 are side views of an ultrasound probe according to a fourth embodiment. FIG. 11 is a diagram of an ultrasound probe 100 viewed in the same direction as FIG. 3, and FIG. 12 is a diagram of the ultrasound probe 100 viewed in the same direction as FIG. 4. In the ultrasound probe 100, the adjustment layer 106 is arranged between the piezoelectric element 101 and the FPC layer 105. The adjustment layer 106 includes the cut surface 106a that is cut by a blade that cuts the piezoelectric element 101. The cut surface 106a is a surface that is formed by being cut by the blade and is a groove.

According to the fourth embodiment, the adjustment material contained in the adjustment layer 106 is a material for performing dressing or pre-cutting on the blade; therefore, when at least a part of the adjustment layer 106 is cut by the blade and the cut surface 106a is formed, dressing or pre-cutting is performed on the blade. As a result, it is possible to improve the cutting performance of the blade and a yield rate of the ultrasound probe 100 to be manufactured. Furthermore, it is not needed to replace a processing target work with a dressing board or a pre-cutting board; therefore, a movement distance of the blade in the cutting direction is not increased, so that it is possible to prevent an increase in a processing time and improve production efficiency.

Moreover, according to the fourth embodiment, the adjustment layer 106 is laminated so as to come into contact with the FPC layer 105, and the adjustment material contained in the adjustment layer 106 performs dressing or pre-cutting on the blade immediately after resin that is likely to cause clogging of the blade or metal of a highly malleable wire is cut, so that it is possible to increase the effect to improve the cutting performance of the blade.

First Modification

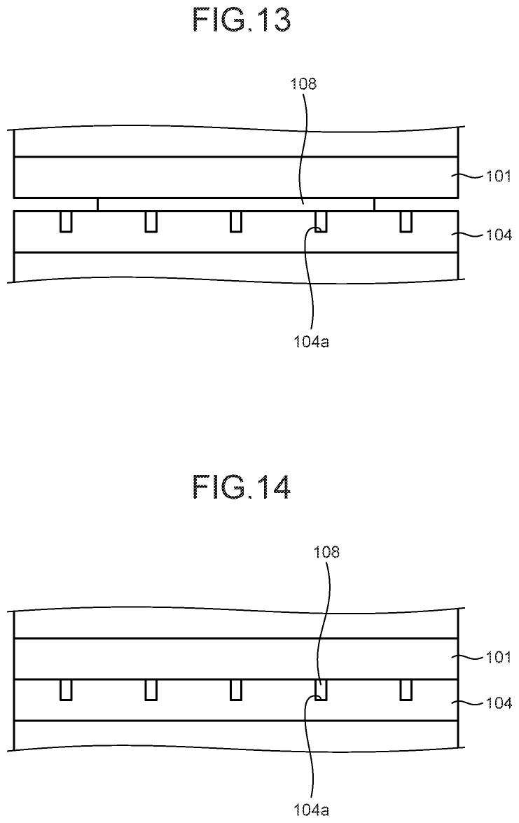

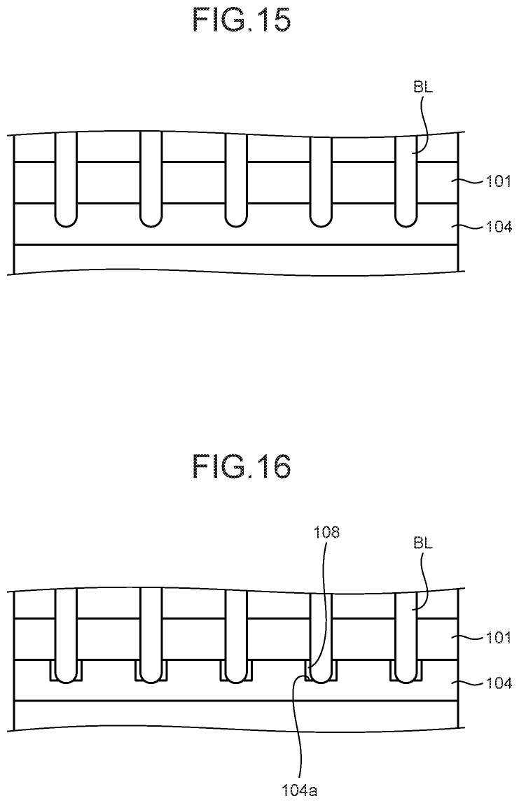

FIG. 13 to FIG. 15 are diagrams illustrating a state in which an ultrasound probe according to a first modification is manufactured. FIG. 13 to FIG. 15 are partial side views of the ultrasound probe. The dematching layer 104 may include grooves 104a as illustrated in FIG. 13. If an adhesive 108 for bonding the piezoelectric element 101 and the dematching layer 104 is applied and the piezoelectric element 101 and the dematching layer 104 are bonded together as illustrated in FIG. 14, the adhesive 108 is put into the grooves 104a as illustrated in FIG. 14, so that a thickness of the adhesive 108 is extremely reduced and the piezoelectric element 101 and the dematching layer 104 are closely attached to each other. Furthermore, as illustrated in FIG. 15, the grooves 104a in each of which the adhesive 108 is put is cut by the blade BL. In this manner, it may be possible to closely attach the piezoelectric element 101 and the dematching layer 104 to each other by forming the grooves 104a in the dematching layer 104. Furthermore, the adhesive 108 may be configured to include an adjustment material and the adhesive 108 may be used as an adjustment layer.

Second Modification

FIG. 16 is a diagram illustrating a state in which an ultrasound probe according to a second modification is manufactured. As illustrated in FIG. 16, a width of each of the grooves 104a may be slightly larger than a width of the blade BL. In this case, as illustrated in FIG. 16, after cutting by the blade BL is performed, a part of the adhesive 108 that has been put into the grooves 104a remains, so that it is possible to more strongly bond the piezoelectric element 101 and the dematching layer 104.

Third Modification

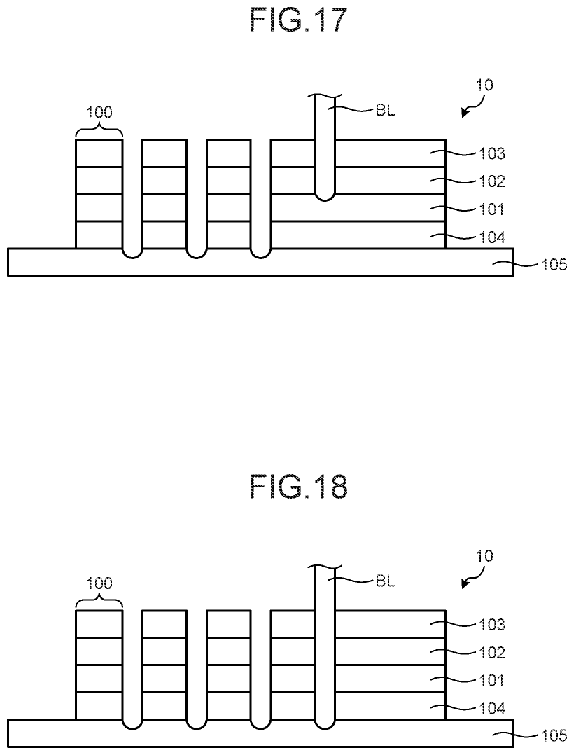

FIG. 17 and FIG. 18 are diagrams illustrating a state in which an ultrasound probe according to a third modification is manufactured. As illustrated in FIG. 17, at first cutting, the second acoustic matching layer 103 and the first acoustic matching layer 102 are cut. The second acoustic matching layer 103 and the first acoustic matching layer 102 are soft layers and therefore cut by the blade BL with large abrasive grains. Subsequently, as illustrated in FIG. 18, the piezoelectric element 101 and the dematching layer 104 are cut. The piezoelectric element 101 and the dematching layer 104 are rigid layers and therefore cut by the blade BL with small abrasive grains. In this manner, by performing cutting several times while changing a size of the abrasive grains of the blade BL, it is possible to prevent the ultrasound probe 10 that is a work from being damaged during processing. By performing processing as described above, surface roughness of the cut surface that is formed by cutting the second acoustic matching layer 103 and the first acoustic matching layer 102 is larger than surface roughness of a cut surface that is formed by cutting the piezoelectric element 101 and the dematching layer 104.

Fourth Modification

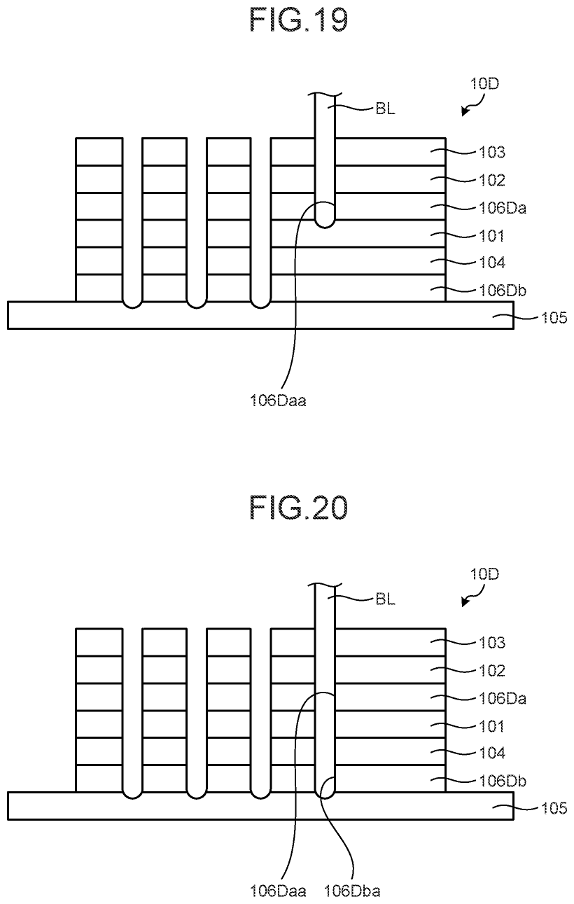

FIG. 19 and FIG. 20 are diagrams illustrating a state in which an ultrasound probe according to a fourth modification is manufactured. As illustrated in FIG. 19 and FIG. 20, an ultrasound probe 10D includes two adjustment layers, that is, an adjustment layer 106Da and an adjustment layer 106Db. The adjustment layer 106Da and the adjustment layer 106Db include adjustment materials that improve cutting performance of the blade that cuts the piezoelectric element 101.

As illustrated in FIG. 19, at first cutting, the second acoustic matching layer 103, the first acoustic matching layer 102, and the adjustment layer 106Da are cut. The second acoustic matching layer 103 and the first acoustic matching layer 102 are soft layers and therefore cut by the blade BL with large abrasive grains. Further, a cut surface 106Daa that is a flat surface is formed on the adjustment layer 106Da.

Subsequently, as illustrated in FIG. 20, the piezoelectric element 101, the dematching layer 104, and the adjustment layer 106Db are cut. The piezoelectric element 101 and the dematching layer 104 are rigid layers and therefor cut by the blade BL with small abrasive grains. Further, a cut surface 106Dba that is a flat surface extending along a side surface of the piezoelectric element 101 is formed on the adjustment layer 106Db.

In this manner, by performing cutting several times while changing a size of the abrasive grains of the blade BL, it is possible to prevent the ultrasound probe 10D that is a work from being damaged during processing. Further, the blade BL of a different type is used for each cutting, so that, by forming the adjustment layer 106Da and the adjustment layer 106Db containing the adjustment materials corresponding to the types of the blades BL, it is possible to increase the effect to improve the cutting performance of the blade BL. By performing the processing as described above, surface roughness of the cut surface that is formed by cutting the second acoustic matching layer 103 to the adjustment layer 106Da is larger than surface roughness of the cut surface that is formed by cutting the piezoelectric element 101 to the adjustment layer 106Db.

Meanwhile, in the embodiment as described above, the example has been described in which the acoustic matching layer, the backing layer, and the like are adopted as the adjustment layers, but embodiments are not limited to this example. It may be possible to adopt any layers except for a layer of the piezoelectric element 101 as the adjustment layer by providing the adjustment material in the subject layer. However, it is needed to cut at least a part of the adjustment layer by the blade BL and form the cut surface at the time of cutting by the blade BL.

According to the disclosure, it is possible to realize an ultrasound probe, an ultrasound endoscope, a laminated body, and an ultrasound probe manufacturing method with a good yield rate and good production efficiency.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound probe comprising:
an ultrasound transducer that includes
a piezoelectric element configured to transmit and receive ultrasound waves to and from a subject; and
an adjustment layer that is laminated on the piezoelectric element, the adjustment layer being provided with a cut surface that is cut by a blade configured to cut the piezoelectric element, the adjustment layer including an adjustment material configured to improve cutting performance of the blade.

2. The ultrasound probe according to claim 1, wherein the ultrasound transducer includes an acoustic matching layer that is laminated on the piezoelectric element in a direction in which the piezoelectric element transmits and receives the ultrasound waves, and the acoustic matching layer has acoustic impedance different from the piezoelectric element.

3. The ultrasound probe according to claim 2, wherein the adjustment layer is laminated on the piezoelectric element in the direction in which the piezoelectric element transmits and receives the ultrasound waves.

4. The ultrasound probe according to claim 3, wherein the adjustment layer is laminated so as to be in contact with the acoustic matching layer.

5. The ultrasound probe according to claim 1, wherein the adjustment layer is an acoustic matching layer that is laminated on the piezoelectric element in a direction in which the piezoelectric element applies the ultrasound waves, and the acoustic matching layer has acoustic impedance different from the piezoelectric element.

6. The ultrasound probe according to claim 1, wherein the ultrasound transducer includes a substrate layer including a flexible board electrically connected to the piezoelectric element.

7. The ultrasound probe according to claim 1, wherein the ultrasound transducer includes a backing layer that is formed of a backing material configured to absorb or attenuate ultrasound waves generated by the piezoelectric element.

8. The ultrasound probe according to claim 1, wherein the ultrasound transducer includes a dematching layer that is laminated on the piezoelectric element in a direction opposite to a direction in which the piezoelectric element transmits and receives the ultrasound waves, and the dematching layer has higher acoustic impedance than the piezoelectric element.

9. The ultrasound probe according to claim 1, wherein the adjustment layer is laminated on the piezoelectric element in a direction opposite to a direction in which the piezoelectric element transmits and receives the ultrasound waves.

10. The ultrasound probe according to claim 6, wherein the adjustment layer is laminated on the piezoelectric element in a direction opposite to a direction in which the piezoelectric element transmits and receives the ultrasound waves, and the adjustment layer is laminated so as to be in contact with the substrate layer.

11. The ultrasound probe according to claim 7, wherein the adjustment layer is laminated on the piezoelectric element in a direction opposite to a direction in which the piezoelectric element transmits and receives the ultrasound waves, and the adjustment layer is laminated so as to be in contact with the backing layer.

12. The ultrasound probe according to claim 1, wherein the adjustment layer is a backing layer that is formed of a backing material configured to absorb or attenuate ultrasound waves generated by the piezoelectric element.

13. The ultrasound probe according to claim 1, wherein the piezoelectric element includes a piezoelectric body that is made of a single crystal.

14. The ultrasound probe according to claim 1, wherein the adjustment material is a material configured to perform at least one of dressing and pre-cutting on the blade.

15. The ultrasound probe according to claim 1, wherein the adjustment material is an abrasive grain that is finer than an abrasive grain used in the blade.

16. The ultrasound probe according to claim 1, wherein the adjustment material is made of a material that is harder than an adhesive used in the blade.

17. An ultrasound endoscope comprising:
the ultrasound probe according to claim 1;
an insertion portion including a distal end on which the ultrasound probe is arranged, the insertion portion being configured to be inserted into the subject; and
an operating portion that is arranged on a proximal end side of the insertion portion.

18. A laminated body comprising:
a piezoelectric layer that is made of a piezoelectric material; and
an adjustment material layer that is laminated on the piezoelectric layer and that includes an adjustment material configured to improve cutting performance of a blade configured to cut the piezoelectric layer.

19. An ultrasound probe manufacturing method comprising:
preparing a laminated body including a piezoelectric layer that is made of a piezoelectric material, and an adjustment material layer that is laminated on the piezoelectric layer and that includes an adjustment material configured to improve cutting performance of a blade configured to cut the piezoelectric layer; and
cutting the piezoelectric layer and at least a part of the adjustment material layer by the blade.
